(19) United States
(12) Patent Application Publication
(10) Pub. No.: US 2005/0283316 A1
(43) Pub. Date: Dec. 22, 2005

(54) Silico iterations correlating mass spectrometer outputs with peptides in databases and success of same

(76) Inventor: Isaac J. Hands, Lexington, KY (US)

Correspondence Address: KING & SCHICKLI, PLLC, 247 NORTH BROADWAY, LEXINGTON, KY, 40507, US

(21) Appl. No.: 10/873,572

(22) Filed: Jun. 22, 2004

(51) Int. Cl. G06F 19/00; G01N 33/48; G01N 33/50

(52) U.S. Cl. 702/19

(57) ABSTRACT

Independent of scoring algorithm for matching or correlating mass spectrometer outputs to peptides in database(s), methods for identifying when a scoring algorithm has achieved a successful correlation include identifying criteria indicative of the successful correlation, conducting a plurality of scoring algorithm runs or analyses, and making an in silico determination as to whether the criteria is met. A first analysis occurs with initial parameters while subsequent analyses occur with modified parameters and/or other scoring algorithms. Parameters include spectrum data conditioning parameters applicable to mass spectrometer outputs and/or peptide data conditioning parameters applicable to peptides or their database. Preferred criteria indicating successful correlation include meeting a threshold algorithm score, obtaining a desired peptide coverage percentage or obtaining an amount of spectrum coverage used in matching. De novo sequencing information may also be used. Computer readable media and computing system environments are some embodiments for performing the invention.

### FIELD OF THE INVENTION

The present invention relates to correlating or matching samples analyzed by mass spectrometers to amino acid sequences or peptides in databases of same. In particular, it relates to iteratively performing the correlation in silico, e.g., in a computing system environment, until criteria indicative of a successful sequence or peptide match is met or exceeded.

### BACKGROUND OF THE INVENTION

The art of correlating or matching samples analyzed by mass spectrometers to amino acid sequences or peptides in databases is becoming relatively well known. In general, an unknown sample 10 is submitted to a mass spectroscopy facility 12 for analysis by a mass spectrometer 14 (FIG. 1). Regardless of spectrometry methodology or approach, the output 16 typically embodies a plot of Intensity vs. Mass which represents an entire unknown sample or a fragment thereof. The mass peaks 18 are then compared 20 to calculated masses of a variety of amino acid sequences 22 or peptides in a database 24, such as one of the databases maintained by the National Center for Biotechnology Information (NCBI) as part of the National Institute of Health (NIH) at http://www.ncbi.nlm.nih.gov, for example. Matches between peaks and peptides then serve to identify the unknown sample and advancement of technology and academia occurs. Scoring algorithms that perform the comparison produce a human readable output that ranks the sequence or peptide matches in a hard or soft copy list 26. Depending upon the mass spectroscopy facility, the output-type of the mass spectrometer (e.g., tandem mass spectroscopy MS/MS or MALDI/TOF) and the desired result, human spectroscopy specialists select which scoring algorithm they prefer. Some of the commercially available scoring algorithms performing this function include Mascot, Sequest, Xtandem and SONAR.

Often, however, mass peaks 18 do not precisely conform or exactly match the masses of sequences or peptides in the database 24. As a result, the scoring algorithms use known or proprietary statistical analysis, probabilities or other techniques to assign a numeric value, or algorithm score, indicating the likelihood that a particular mass peak 18 matches a particular amino acid sequence or peptide mass calculated/stored by the database. Problematically, the failure or success of matching an unknown sample to peptides in databases ultimately relies with the human spectroscopy specialist. For example, if a scoring algorithm produces a list that matches five peptides to a given mass peak 18, and the scores for each of the five matches range from number 1 to number 5 (on a scale of number 0 (least) to number 10 (most)), the specialist can conclude that the peptide match having a number 5 score corresponds to the measured mass of the unknown sample and quit the analysis. Alternatively, the specialist can conclude none of the matches have a high enough score and re-submit the mass peak 18 to the scoring algorithm for another scoring run. To avoid reproducing the same exact results, the specialist will alter various parameters of the scoring algorithm. Then, if the specialist likes the score of the subsequent run, they are again free to conclude a match has occurred and quit the analysis. They can also re-submit for still another scoring run and repeat the process. As is often the case, a specialist attempts numerous re-submissions when correlating samples to peptides. Some, however, consider this too heavily dependent on human judgment and time consuming.

Accordingly, a need exists in the art for minimizing human judgments and speeding the process.

### SUMMARY OF THE INVENTION

The above-mentioned and other problems become solved by applying the principles and teachings associated with the hereinafter described methods for iteratively matching or correlating outputs of mass spectrometers to amino acid sequences or peptides in databases of same and indicating successful matches thereof. In general, a software architecture iteratively performs numerous scoring runs, with minimal human intervention and quick processing times, until a successful outcome is achieved. It also does so without regard for a particular scoring algorithm and in an environment requiring numerous changed parameters in a given scoring algorithm, multiplicities of possible scoring algorithms, multiplicities of peptide match tests, and dynamic computer resource availability.

In one embodiment, independent of a particular scoring algorithm, methods for identifying when scoring algorithms achieve successful correlation between mass spectrometer outputs and peptides in databases include (i) identifying criteria indicative of the successful correlation, (ii) conducting a plurality of scoring algorithm runs or analyses, and (iii) making an in silico determination as to whether the criteria is met or not. A first scoring algorithm analysis occurs with initial parameters while subsequent analyses occur with modified parameters and/or other scoring algorithms. Parameters of the invention include, but are not limited to, spectrum data conditioning parameters applicable to mass spectrometer outputs and peptide data conditioning parameters applicable to the peptides or their database. With more specificity, preferred spectrum data conditioning parameters relate to removing low intensity peaks, low mass peaks and/or noise from the output of the mass spectrometer. Preferred peptide data conditioning parameters include selecting taxonomy, indicating modifying masses and/or alternate digestion techniques. Preferred criteria indicating a successful peptide correlation or match include meeting a threshold algorithm score, obtaining a desired peptide coverage percentage or obtaining a threshold amount of spectrum coverage during matching. De novo sequencing information may also be used.

In other aspects, scoring algorithm analyses are iterated until one of three configuration conditions is met. The conditions include the meeting or exceeding of a criterion that indicates a successful peptide match, attempting all possible spectrum and/or data conditioning parameters during the scoring algorithm runs or reaching a computing resource limitation.

Computer readable media and computing system environments having computer executable instructions for executing the foregoing are some specific embodiments for performing the invention. Still other aspects of the invention include displaying and receiving indications from users relative to creating a scoring description of the sample that corresponds to the spectrum and peptide data conditioning parameters and/or the criteria for ascertaining successful peptide matches.

These and other embodiments, aspects, advantages, and features of the present invention will be set forth in the description which follows, and in part will become apparent to those of ordinary skill in the art by reference to the following description of the invention and referenced drawings or by practice of the invention. The aspects, advantages, and features of the invention are realized and attained by means of the instrumentalities, procedures, and combinations particularly pointed out in the appended claims.

### BRIEF DESCRIPTION OF THE DRAWINGS

FIG. 1 is a diagrammatic view in accordance with the teachings of the prior art for correlating a mass spectrometer output with amino acid sequences or peptides in databases of same;

FIG. 2 is a flow chart in accordance with the teachings of the present invention for creating meta-data, including creating a scoring description indicative of spectrum and peptide data conditioning parameters and criteria for successful peptide matches;

FIG. 3 is a block diagram in accordance with the teachings of the present invention indicating a preferred scoring description;

FIG. 4 is a block diagram in accordance with the teachings of the present invention indicating preferred spectrum data conditioning parameters;

FIG. 5 is a block diagram in accordance with the teachings of the present invention indicating preferred peptide data conditioning parameters;

FIG. 6 is a block diagram in accordance with the teachings of the present invention indicating preferred criteria for indicating successful peptide matches;

FIG. 7 is a diagrammatic view in accordance with the teachings of the present invention of an exemplary mass spectrometer output;

FIG. 8 is a flow chart in accordance with the teachings of the present invention indicating the in silico iterative correlation of a mass spectrometer output to amino acid sequences or peptides in a database and the successful correlation thereof;

FIG. 9 is a block diagram in accordance with the teachings of the present invention indicating preferred configuration conditions;

FIG. 10 is a diagrammatic view in accordance with the teachings of the present invention of a representative computing system environment in which the invention may be practiced; and

FIG. 11 is a diagrammatic view in accordance with the teachings of the present invention of a representative software abstraction useful in the operating environment of FIG. 10.

### DETAILED DESCRIPTION OF THE PREFERRED EMBODIMENTS

In the following detailed description of the preferred embodiments, reference is made to the accompanying drawings that form a part hereof, and in which is shown by way of illustration, specific embodiments in which the invention may be practiced. These embodiments are described in sufficient detail to enable those skilled in the art to practice the invention, and it is to be understood that other embodiments may be utilized and that process, hardware, software and/or other changes may be made without departing from the scope of the present invention. The following detailed description is, therefore, not to be taken in a limiting sense, and the scope of the present invention is defined only by the appended claims and their equivalents. In accordance with the present invention, in silico methods for iteratively matching or correlating outputs of mass spectrometers to amino acid sequences or peptides in a database of same are hereinafter described. So too are the indications of successful matches thereof.

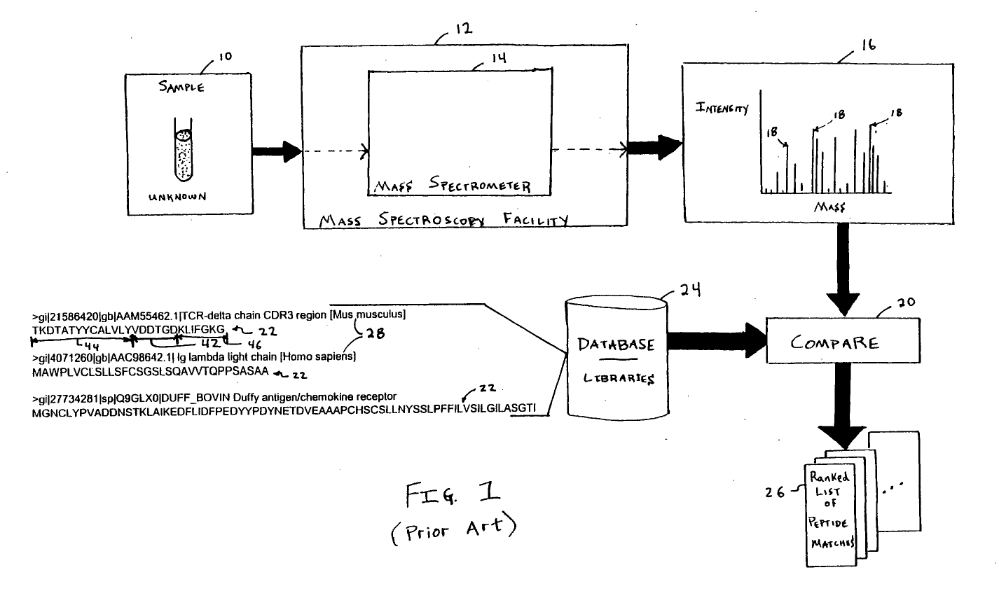

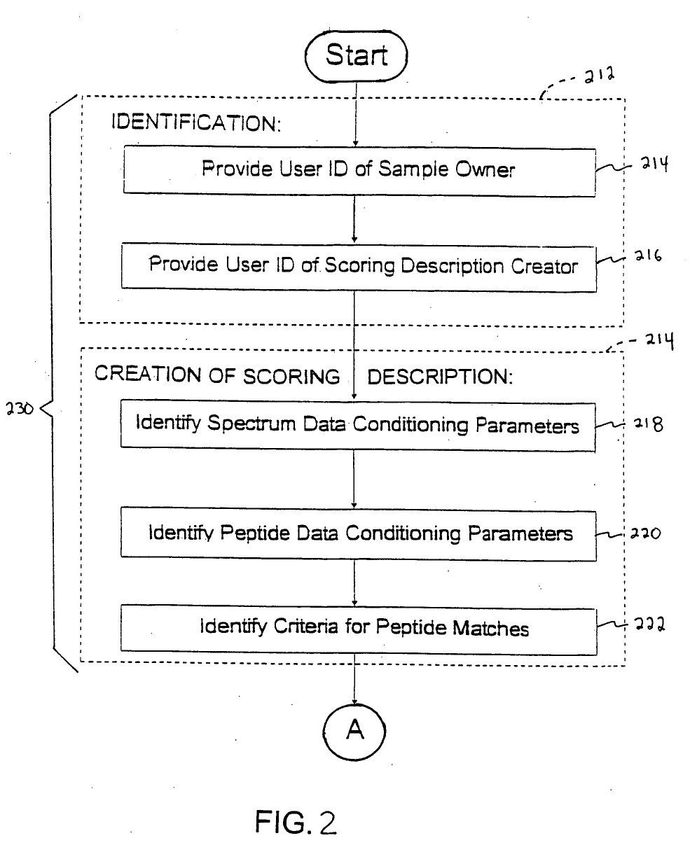

As a preliminary matter regarding convention, the invention sometimes expressly recites both amino acid sequences and peptides and at other times only mentions one and not the other. The invention at all times, however, relates to both amino acid sequences and peptides despite the presence of only one descriptor. In silico and in a computing or operating system environment may also be treated as interchangeable environments in the specification and claims. Also, discussion of a criterion or criteria having been met will simultaneously mean the criterion or criteria has been met and/or exceeded despite the singular existence of the term “met.” Lastly, the invention will be initially described as a methodology (FIGS. 2-9) and then as an apparatus or abstraction, such as in the context of software or computer executable instructions in a computing system environment. In either instance, reference will sometimes be made to FIG. 1 for it has applicability to the instant invention as the genesis thereof.

With reference to FIG. 2, when a partially known, unknown or other sample 10 (FIG. 1) is submitted to a mass spectroscopy facility for analysis, meta-data 230 is created. As part thereof, it is identified 210 and a scoring description is created 212. During identification, user identification of the sample owner is provided 214 as is user identification of the creator of the below-described scoring description 216. In this manner, administrative matters and tracking within the facility and between the facility and the owner/creator can be maintained. Naturally, the owner and creator may be the same person(s) or legal entities. Although not shown, identification 212 may additionally include access control lists for results, delivery instructions or other.

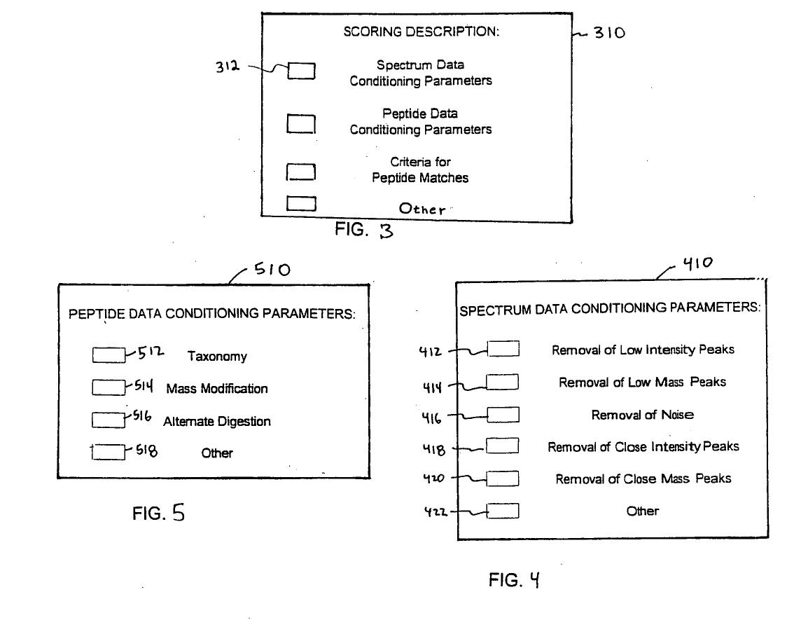

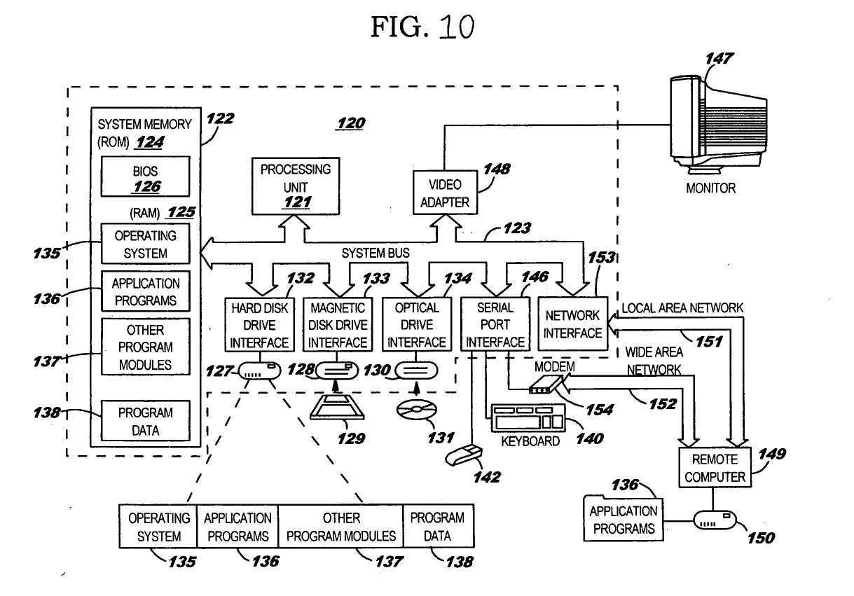

During creation of the scoring description, the creator or operating system indicates or otherwise identifies spectrum data conditioning parameters 218, peptide data conditioning parameters 220 and criteria corresponding to a successful peptide match 222. In general, these items together define a range that the invention will use to analyze the sample and iteratively correlate or match a mass spectrometer output to peptides in databases. They will also enable the reporting of successes thereof. In various embodiments, the creator provides the scoring description directly to the facility, the operating system provides it if the creator has no preference or is unable to provide it, or a hybrid whereby the information is obtained from both the creator and operating system. Although primarily described hereafter in the context of a creator indicating their preference(s), the invention at all times relates to the operating system providing it or a creator/operating system hybrid. In a preferred embodiment, the creator provides it electronically or the facility enters it electronically after verbal, paper or other non-electronic submission. In one instance, queries may be displayed directly to the creator via a monitor (FIG. 10). Responses thereto may be indicated or selected via a keyboard and/or other pointing device (FIG. 10) and permanently, semi-permanently or fleetingly stored in memory for later processing. Queries may also come in the form of sequential pages of display as indicated in FIGS. 3-5, for example.

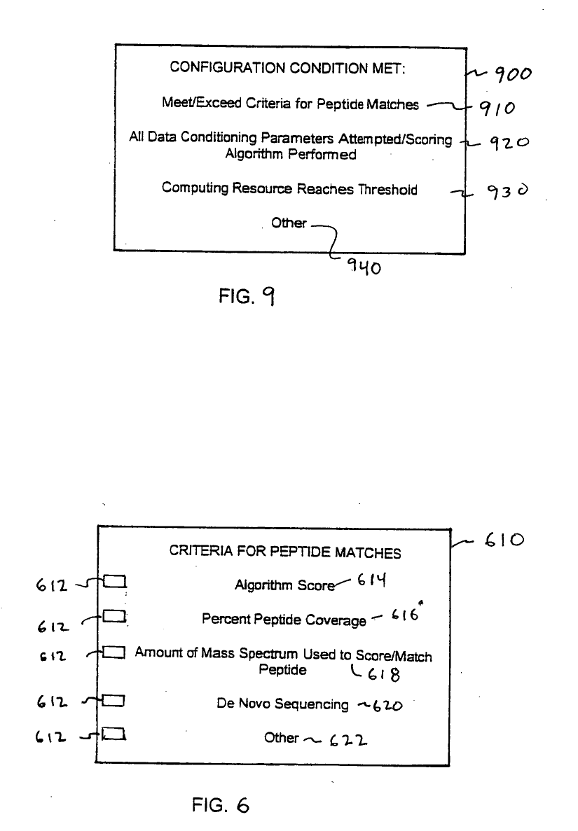

With more specificity, FIG. 3 depicts a representative scoring description page 310, in the form of a menu, which a creator may indicate a selection to by checking a box 312 with a pointing device, for example. Specific menu items include any of the spectrum or peptide data conditioning parameters or the criteria for peptide matches. Selecting one of these menu items, in turn, preferably takes the creator to a subsequent page indicated in FIGS. 4, 5 and 6, for example.

As is known, the sample itself may be of any origin and embody a peptide, a protein or other to-be-analyzed substance. It may also have previously undergone purification and/or enzymatic digestion as is also known. In such instances, the creator would provide this information to the facility and include it as part of the scoring description under the “Other” menu item of page 310, for example. “Other” may also embody known or hereinafter discovered information useful in creating a scoring description.

In FIG. 4, a spectrum data conditioning parameter page is given as a menu 410. In a preferred embodiment, spectrum data conditioning parameters include those items to be applied to mass spectrometer outputs. Representative examples include indicating how-to-remove low intensity peaks, low mass peaks, noise or the like.

As an example, consider the output 700 of a mass spectrometer in FIG. 7. In such figure, intensity values are given along the vertical axis while mass is given along the horizontal axis. The output 700 may be the result of any whole or fragmented sample processed by a mass spectrometer according to any variety of methodologies, such as MS/MS. Also, the output represents the relative abundances of ions produced in an ion source as a function of their mass-to-charge ratios as is well known in the art and, thus, not described herein in further detail. Other detail on the subject, however, can be found in Tandem Mass Spectrometry: a Primer, Edmond de Hoffman, Journal of Mass Spectrometry, vol. 31, pp. 129-137 (1996), for example, and is incorporated herein by reference. In one instance, a low intensity peak corresponds to values less than 20, such as peak 127.81 given as element 710. In another instance, a low mass peak corresponds to any mass less than 300, such as peaks 297.14, 283.74 and 127.81, given as elements 712, 714 and 710, respectively. Noise, on the other hand, represents any peak not having a mass number expressly recited. In turn, a creator making a scoring description for the sample will select “removal of low intensity peaks” by checking box 412. In turn, this allows them to indicate or otherwise identify those peaks having intensities of “less than 20,” for example. Similarly, by checking box 414, the creator indicates “less than 300,” for example, for removing low mass peaks and so on for removing noise via box 416. Alternatively, other methodologies to indicate these values include providing an indication of an upper and lower limit and an increment value. In such instance, a creator might enter 10 as a lower limit or start value for removing low intensity peaks and an upper limit or end value of 20. They may also provide an increment value of some number befitting a range between 10 and 20, such as 1, 2 or 5. Each of these techniques, however, will be discussed below in greater detail during the scoring algorithm analysis. Still alternatively, as before, the creator may have no preference and the operating system or creator/operating system hybrid will supply one, some or all of the spectrum data conditioning parameters.

Other spectrum data conditioning parameters might include removing “close intensity peaks” or “close mass peaks” by checking boxes 418 or 420. As an example of these, consider mass peaks 716 and 718 having masses of 541.27 and 542.08. Not only can skilled artisans consider these two peaks close in intensity but also close in mass. Thus, if so desired, processing of the mass spectrum output can remove one or the other of these peaks.

Still other spectrum data conditioning parameters in the meta-data include, but are not limited to, a minimum parent ion mass, a minimum fragment mass, the mass tolerance to consider for peptide matches (e.g., how close/how many Daltons does a mass peak 18 need to be to a calculated mass of a peptide in a database to be considered), and signal-to-noise ratios. These or other spectrum data conditioning parameters can be entered via the functionality of box 422 for the menu item “Other.”

Peptide data conditioning parameters, in FIG. 5, representatively correspond to taxonomy 512, mass modification 514, alternate digestion 516 or other 518 and may appear to a creator as items on a menu of page 510. In general, peptide data conditioning parameters are those that will be applied to the database 24 (FIG. 1) of peptides or individual peptides 22 (FIG. 1) during the scoring algorithm analyses described below.

With more specificity, taxonomy includes an indication of a creator's preference to compare their sample to various classifications within the databases. Taxonomy will apply to single organisms or a collection of organisms of suspected origin and a description on how to walk the taxonomic tree to find matches. An example of taxonomy can be seen in FIG. 1 as element 28. Well known taxonomies include H. sapiens (Homo sapiens), M. musculus (mus musculus), S. cerevisiae, C. elegans, D. melanogaster or the like. Aside from the creator's indication of a particular taxonomy, the operating system/meta-data may additionally provide related or logical additions thereto without further indication from the creator. For example, if a creator specifies taxonomy as mus musculus, the meta-data may additionally add rattus norvegicus or other rodentia as potential places to find a peptide match. Alternatively, if Homo sapiens are identified, the meta-data may also consider human bacteria because samples 18 (FIG. 1) of human tissue often contain bacterial infections.

Mass modification includes an indication of a creator's preference to modify amino acid sequences 22 (FIG. 1) with various other masses to further expand the search to find peptide matches. Well known examples of this include isotope labeling, phosphorylation, acetylation, biotinylation, alkylation, palmitoylation, glycosylation, ribosylation, hydroxylation, methylation, or the like. Alternate digestion relates to a creator's preference to consider alternate methods of digestion such as with a proteolytic enzyme such as trypsin. Other examples include formic acid, CNBr, chymotrypsin, PepsinA or the like.

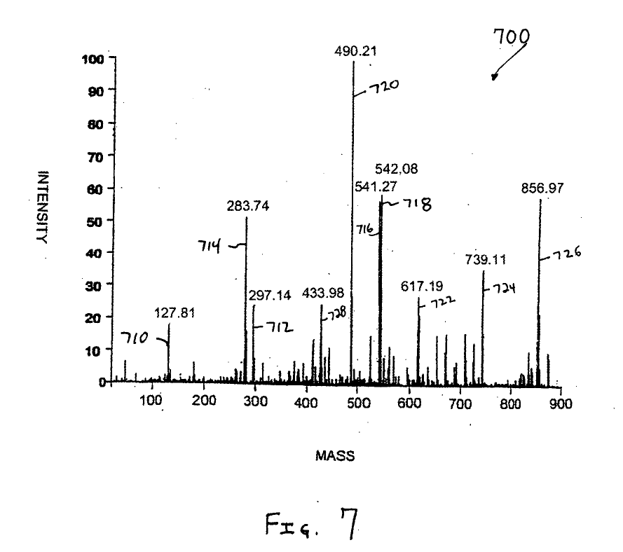

With reference to FIG. 6, scoring description information of the meta-data further includes a creator's indication of criteria for considering a peptide match run by a scoring algorithm a success or not. Similar to other meta-data, the criteria for peptide matches can be displayed as a page 610 with check blocks 612 that users select to receive additional pages for providing specific content therein. It can also be configured such that the operating system provides the criteria if the creator has no preference, such as by selecting criteria of a previous result or basing criteria on system-wide settings, or via the functionality of the creator/operating system hybrid. In a preferred embodiment, the criteria for indicating successful peptide matches will include the individual criterion of algorithm score 614, percent peptide coverage 616, amount of mass spectrum used to score/match peptide 618, de novo sequencing 620 and other 622.

Algorithm score 614 can embody many different concepts. In one aspect, it can embody a particular minimum score that a given scoring algorithm uses to grade its peptide matches. For example, if a scoring algorithm uses a scale of number 0 to number 10 to indicate the level of success of peptide matches, the creator may indicate a successful match if that particular scoring algorithm returns a number of 8 or higher. In another scoring algorithm, having a scale of 0% to 100% to indicate likelihood that peptide matches are accurate, the creator may provide a minimum acceptable score of 75%. Skilled artisans can, of course, think of other suitable examples.

The percent peptide coverage 616 relates to an acceptable minimum amount of usage of a given peptide. For example, if the scoring algorithm returns single or plural matches to a portion 42 (FIG. 1) of a given peptide; consider whether portion 42 relates to a sufficient percentage of the entire peptide having portions 42, 44 and 46. In such instance, a creator may desire to specify that a success occurs if the single or plural matches relate to more than 50 percent of the entire peptides. Of course, any number or percentage may be specified depending upon preference.

On the other hand, the amount of mass spectrum used to score/match peptides 618 relates conceptually to the inverse or reciprocal of percent peptide coverage 616 and creators can also indicate their preference to this criterion. For example, consider the output 700 of FIG. 7. In the event a scoring algorithm only returns peptide matches for mass peak 720 (having a mass of 490.21), skilled artisans readily observe that this only represents one-tenth of the spectrum, (the other mass peaks include 710, 712, 714, 716, 718, 722, 724, 726 and 728). In such instance, a creator may want to indicate their preference here as requiring more than 50% of the spectrum be used in obtaining matches. In this example, since ten mass peaks (710, 712, 714, 716, 718, 720, 722, 724, 726, and 728) are available in the spectrum, more than 50% would require having peptide matches for six or more mass peaks. Any number or percentage may be specified depending upon preference.

De novo sequencing 620 will be another criterion used to determine when the best peptide match has been found. As presently contemplated, de novo sequencing will directly compare the mass peaks of the spectrometer output to the masses of the twenty or so amino acids, actually available in life, and determine if a match exists. Preferably, all of the possible de novo peptide sequences will be compared against the peptide sequences resulting from the scoring runs by a sequence alignment algorithm. If a peptide sequence from the scoring run matches a de novo sequence with a specified minimum alignment score, then this criterion will be satisfied.

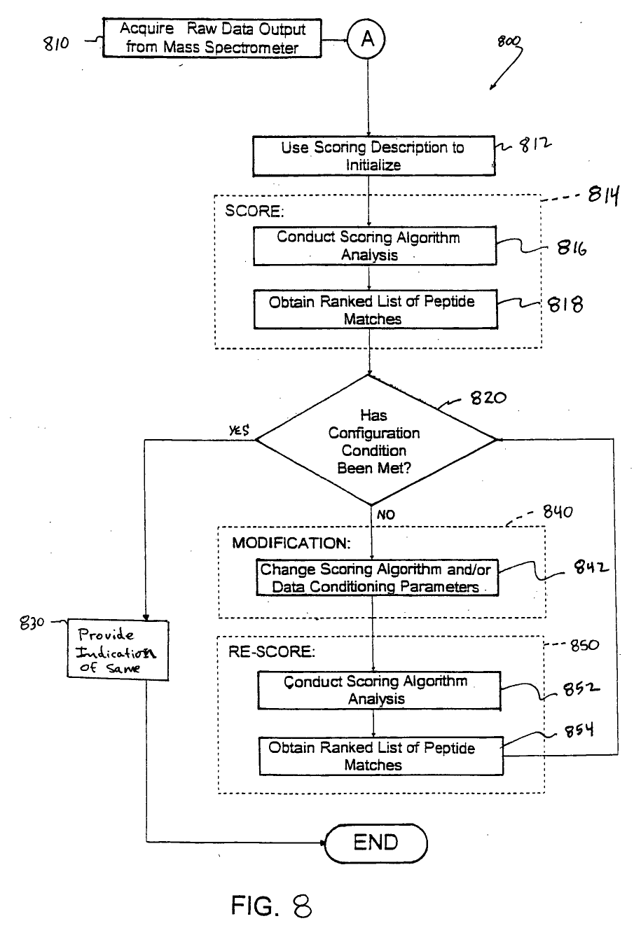

Once a creator or facility completes the information for the meta-data 230, especially the scoring description 214, iterative matching or correlation of mass spectrometer outputs to amino acid sequences or peptides in a database of same can be accomplished with great speed and without excessive human (spectroscopy specialist) intervention. Success of the correlation can also occur relatively quickly. Again, skilled artisans will appreciate the completion of the meta-data may alternately occur as the result of the operating system or creator/operating system hybrid supplying the information. With reference to FIG. 8, the raw data output of a mass spectrometer is acquired or obtained at step 810. As in the prior art, the output typifies a plot of Intensity vs. Mass for a given unknown sample 18 (FIG. 1) and is acquired by a mass spectroscopy facility as previously discussed. A representative example, again, corresponds to the output 700 shown in FIG. 7.

Thereafter, or simultaneously with step 810, the scoring description of the meta-data is used to initialize 812 an initial or first to-be-run scoring algorithm. Preferably, the initialization includes selecting one or more of the spectrum and peptide data conditioning parameters made by the creator in their scoring description and providing or making the parameter(s) available for use by the first scoring algorithm. As an example, if the data conditioning parameters included a start value, an ending value and an increment value according to a prior example, the initialization herein would automatically use the start value as the initial parameter. In the event the parameters were entered as “less than 20” for removing low intensity peaks 412 according to another prior example, the initialization 812 could then either have a subroutine that first uses “20” and then decrements from there. Alternatively, it could initialize with an intensity of “10” and then increment the values until reaching the creator's limit of “20.” Skilled artisans are also able to contemplate other relevant examples.

Once initialized, a first scoring run is conducted 814 using the initial parameter(s). Specifically, a scoring algorithm analysis is conducted 816 and a ranked list of peptide matches is obtained 818. The conducting of this scoring algorithm analysis is done in the same general accord with the prior art, yet may be undertaken with any scoring algorithm presently available or any hereafter invented. In the prior art, however, it is this last step that causes the introduction of a human spectroscopy specialist (mass spectrometrist) into the analysis which slows the process and causes subjectivity. In contrast, the instant invention does not provide the ranked list of peptide matches to a human. Instead, an in silico operation analyzes them to determine whether a configuration condition is met 820.

Referring to FIG. 9, the meeting of a configuration condition 900 occurs in one of three instances. Specifically, it occurs if one or more of the criterion of the criteria for peptide matches (610) is met or exceeded 910. It occurs if all the spectrum (410) and peptide (510) data conditioning parameters specified by the creator (alt: operating system or creator/operating system hybrid) have been attempted in a scoring algorithm analysis run per each of the possible scoring algorithms 920. It occurs if the operating environment or computing resources, such as memory or processor usage, reach some limiting threshold 930, such as the lack of available memory or too taxing processing. In other embodiments, skilled artisans can enter other reasons 940 for concluding a configuration condition is met. Of course, none, one or more-than-one configuration condition can be met at any given time.

Referring back to FIG. 8, if one of the configuration conditions is met at 820, the process ends and an indication of same is provided at step 830. Of course, this will also end the scoring or running of scoring algorithms. Since it is unlikely that a configuration condition 900 will be met upon conducting a first scoring run 814, a modification 840 and re-scoring run 850 usually occur.

A modification 840, in turn, further includes changing the scoring algorithm at step 842 to another scoring algorithm and/or changing one, some or all of the initial parameters (e.g., step 812) into modified data conditioning parameters. A re-scoring run 850 contemplates conducting another scoring algorithm analysis 852 with the modified parameters or a new algorithm and obtaining another ranked list of peptide matches 854.

With more specificity, the modification of an initial parameter into a modified parameter may simply consist of changing the start value of a spectrum data conditioning parameter by an amount equivalent to the increment value as discussed in a previous example. It may also consist of removing noise from the output of the mass spectrometer whereas noise was previously included in the prior scoring run at step 814. Those skilled in the art can readily figure other modifications and no further discussion is necessary. Alternatively, it may consist of changing a peptide data conditioning parameter, such as examining a taxonomy other than that originally examined in the scoring run at step 814. It may also consist of adding a mass modification or examining an alternate digestion. Like the spectrum data conditioning parameters, skilled artisans can readily figure other modifications and no further discussion is necessary.

Modification 840 can also take the form of switching scoring algorithms altogether. From the background, some of the commercially available scoring algorithms and their software include Mascot, Sequest, Xtandem and SONAR. U.S. Pat. Nos. 5,538,897 and 6,271,037, incorporated herein by reference, also teach patented methods. Also, Mass Spectrometry and the Age of the Proteome, John R. Yates, Journal of Mass Spectrometry, vol. 33, pp. 1-19 (1998), incorporated herein by reference, provides information on correlating mass spectrum outputs to known sequences in databases. In the context of the invention, if a first scoring run 814 occurs with Mascot, a subsequent re-scoring run 850 could then occur with SONAR. The invention, however, is not limited to any particular scoring algorithm and could occur with other known or hereinafter programs. Of course, switching or changing scoring algorithms would also likely require an initialization of sorts to accomplish a first run with the new algorithm.

After the modification, and upon obtaining a ranked list of peptide matches 854 from the subsequent scoring algorithm analysis 852, the invention again examines whether a configuration condition is met 820. If a configuration condition is in fact met, the process 800 ends and indication of same is provided at step 830. If not, modification 840 and re-scoring 850 continue until eventually a configuration condition becomes met. As before, preferred configuration conditions include meeting/exceeding one or more criterion of the criteria for peptide matches 910, attempting scoring runs of all possible data conditioning parameters per scoring algorithm 920, reaching a computing resource threshold 930 or other 940. Skilled artisans should now recognize the invention accomplishes numerous scoring algorithm analyses with minimal human intervention which greatly speeds the process. Also, scoring algorithm analysis is not limited to any one of the popular commercial packages which better serves sample owners in ascertaining an understanding of the peptides in their samples. Still other advantages are easily recognized by those of skill in the art.

In alternate embodiments, pluralities of re-scoring runs 850 can occur simultaneously with one another and need not occur sequentially as indicated. Pluralities of initial scoring runs 814 can also occur simultaneously with one another.

Turning now to the physical implementation of the invention, it is expected that users will likely accomplish some aspect of the methods in a computing system environment. As such, FIG. 10 and the following discussion are intended to provide a brief, general description of a suitable computing environment in which either the structure or processing of embodiments may be implemented. Since the following may be computer implemented, particular embodiments may range from computer executable instructions as part of computer readable media or memory to hardware used in any or all of the following depicted structures. Implementation may additionally be combinations of hardware and computer executable instructions. Further, implementation may occur in an environment not having the following computing system environment so the invention is only limited by the appended claims and their equivalents.

When described in the context of computer readable media or memory having computer executable instructions, it is denoted that the instructions include program modules, routines, programs, objects, components, data structures, patterns, trigger mechanisms, signal initiators, etc. that perform particular tasks or implement particular abstract data types upon or within various structures of the computing environment. Executable instructions exemplarily comprise instructions and data which cause a general purpose computer, special purpose computer, or special or general purpose processing device to perform a certain function or group of functions.

The computer readable media, where scoring algorithms, data conditioning parameters, scoring description, criteria for peptide matches or other aspects of the invention may directly reside, can be any available media which can be accessed by a general purpose or special purpose computer or device. By way of example, and not limitation, such computer readable media can comprise RAM, ROM, EEPROM, CD-ROM or other optical disk storage devices, magnetic disk storage devices or any other medium which can be used to store the desired executable instructions or data fields and which can then be accessed. Combinations of the above should also be included within the scope of the computer readable media. For brevity, computer readable media having computer executable instructions may sometimes be referred to as software or computer software.

With reference to FIG. 10, an exemplary system for implementing the invention includes a general purpose computing device in the form of a conventional computer 120. The computer 120 includes a processing unit 121, a system memory 122, and a system bus 123 that couples various system components including the system memory to the processing unit 121. The system bus 123 may be any of the several types of bus structures including a memory bus or memory controller, a peripheral bus, and a local bus using any of a variety of bus architectures. The system memory 122, where scoring algorithms, data conditioning parameters, scoring description, criteria for peptide matches or other aspects of the invention may directly reside, includes read only memory (ROM) 124 and a random access memory (RAM) 125. A basic input/output system (BIOS) 126, containing the basic routines that help to transfer information between elements within the computer 120, such as during start-up, may be stored in ROM 124. The computer 120 may also include a magnetic hard disk drive 127, a magnetic disk drive 128 for reading from and writing to removable magnetic disk 129, and an optical disk drive 130 for reading and writing to an optical disk 131 such as a CD-ROM or other optical media. The hard disk drive 127, magnetic disk drive 128, and optical disk drive 130 are connected to the system bus 123 by a hard disk drive interface 132, a magnetic disk drive interface 133, and an optical drive interface 134, respectively. The drives and their associated computer-readable media provide nonvolatile storage of computer readable instructions, data structures, program modules and other data for the computer 120.

Although the exemplary environment described herein employs a hard disk, a removable magnetic disk 129 and a removable optical disk 131, it should be appreciated by those skilled in the art that other types of computer readable media exist which can store data accessible by a computer, including magnetic cassettes, flash memory cards, digital video disks, removable disks, Bernoulli cartridges, random access memories (RAMs), read only memories (ROM), downloads from the internet and the like. Other storage devices are also contemplated as available to the exemplary computing system. Such storage devices may comprise any number or type of storage media including, but not limited to, high-end, high-throughput magnetic disks, one or more normal disks, optical disks, jukeboxes of optical disks, tape silos, and/or collections of tapes or other storage devices that are stored off-line. In general however, the various storage devices may be partitioned into two basic categories. The first category is local storage which contains information that is locally available to the computer system. The second category is remote storage which includes any type of storage device that contains information that is not locally available to a computer system. While the line between the two categories of devices may not be well defined, in general, local storage has a relatively quick access time and is used to store frequently accessed data, while remote storage has a much longer access time and is used to store data that is accessed less frequently. The capacity of remote storage is also typically an order of magnitude larger than the capacity of local storage. In either instance, the storage needed for the invention may occur remotely or locally.

A number of program modules may be stored on the hard disk 127, magnetic disk 129, optical disk 131, ROM 124 or RAM 125, including but not limited to an operating system 135, one or more application programs 136, other program modules 137, and program data 138. Such application programs may include, but are not limited to, word processing programs, drawing programs, games, viewer modules, graphical user interfaces, image processing modules, intelligent systems modules or other known or hereinafter invented programs. It may especially include proprietary scoring algorithms previously discussed. A user enters commands and information into the computer 120 through input devices such as keyboard 140 and pointing device 142. Other input devices (not shown) may include a microphone, joy stick, game pad, satellite dish, scanner, camera, personal data assistant, or the like. These and other input devices are often connected to the processing unit 121 through a serial port interface 146 that couples directly to the system bus 123. It may also connect by other interfaces, such as parallel port, game port, firewire or a universal serial bus (USB). It could even occur wirelessly via RF, Bluetooth, WiFi or the like.

A monitor 147 or other type of display device connects to the system bus 123 via an interface, such as a video adapter 148. As before, the monitor is one mechanism for displaying queries to a creator during their entry of the meta-data, especially the scoring description. The pointing device and keyboard preferably combine as the mechanism for responding to the queries which ultimately become used during the initial scoring run 814 and subsequent runs 850. In addition to the monitor, the computing system environment may also include other peripheral output devices, such as speakers, printers, scanners, etc. (not shown) that often connect via a parallel port interface (not shown), the serial port interface, USB, Ethernet or other ports.

During use, the computer 120 may operate in a networked environment using logical connections to one or more other computing configurations, such as a remote computer 149. Despite its name, the remote computer 149 may broadly be a personal computer, a server, a router, a network PC, a peer device or other common network node. It will also typically include many or all of the elements described above relative to the computer 120 although only a memory storage device 150 having application programs 136 has been illustrated. It may also be the remote source where scoring algorithms, data conditioning parameters, scoring description, criteria for peptide matches and/or other aspects of the invention reside. Obviously, the more remote computers 149 available, the larger/faster the computing power of the invention. Naturally, more computing resources will lessen the possibility of a condition configuration 900 (FIG. 9) being met at step 820 (FIG. 8) by a computing resource reaching a limiting threshold 930 (FIG. 9) before a configuration condition is met that corresponds to the meeting/exceeding a criterion of the criteria for peptide matches. Contemplated embodiments even consider situations where local computers have access to the remote computer via a monthly or pay-per-use subscription. Some of the typical logical connections between the computer 120 and the remote computer 149 include a local area network (LAN) 151 and/or a wide area network (WAN) 152 that are presented here by way of example and not limitation. Such networking environments are commonplace in offices with enterprise-wide computer networks, intranets and the Internet, but may also be adapted for use in a mobile environment at multiple fixed or changing locations.

When used in a LAN networking environment, the computer 120 is connected to the local area network 151 through a network interface or adapter 153. When used in a WAN networking environment, the computer 120 typically includes a modem 154, T1 line, satellite or other means for establishing communications over the wide area network 152, such as the Internet. The modem 154, which may be internal or external, is connected to the system bus 123 via the serial port interface 146. In a networked environment, program modules depicted relative to the computer 120, or portions thereof, may be stored in the local or remote memory storage devices and may be linked to various processing devices for performing certain tasks. It will be appreciated that the network connections shown are exemplary and other means of establishing a communications link between the computers may be used. Moreover, those skilled in the art will appreciate that the invention may be practiced with other computer system configurations, including host devices in the form of hand-held devices, multi-processor systems, micro-processor-based or programmable consumer electronics, network PCs, minicomputers, computer clusters, main frame computers, and the like.

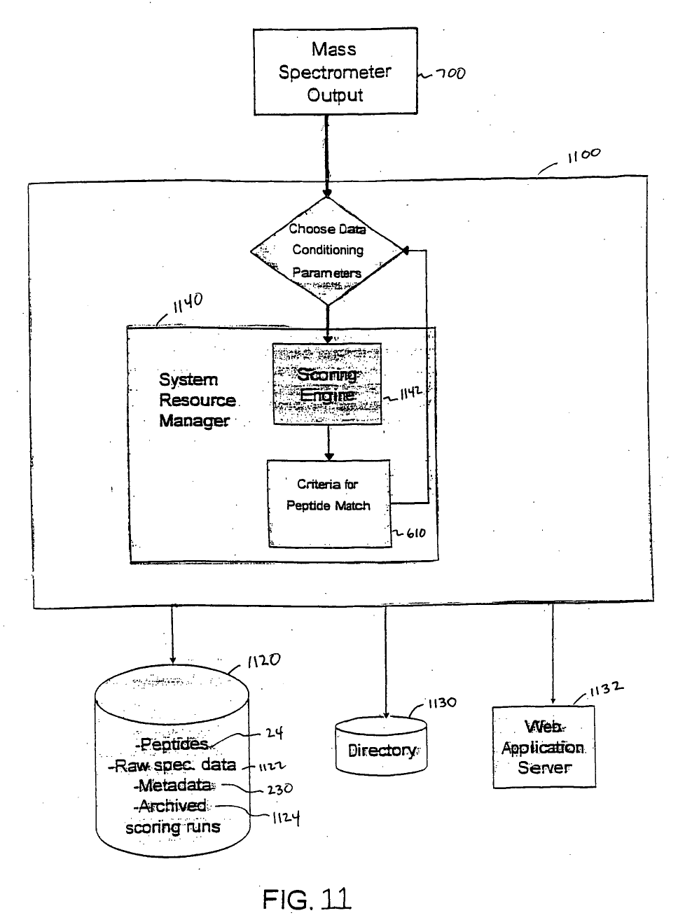

With reference to FIG. 11, an abstraction of the invention includes computer executable instructions 1100 that interface between the output 700 of the mass spectrometer and stored information in a relational database 1120. The relational database includes, but is not limited to, databases of peptides 24, raw data 1122 corresponding to the output 700 of the mass spectrometer, the meta-data 230 and any previous or archived scoring algorithm runs 1124. The computer executable instructions may also interface with a directory 1130 employing a common interface, such as LDAP. The directory preferably includes, but is not limited to, information such as the identification aspect of the scoring description and/or user preferences/profiles that become established over time. Further, the computer executable instruction may optionally interface with a web application server 1132, such as Apache, IIS or Tomcat to display results. The relational database, the directory and web application server, along with messaging capabilities, are presently available in major computing platforms known as J2EE, .Net and WebObjects.

Additionally, the computer executable instructions include a system resource manager 1140 that includes a scoring engine 1142 and the criteria for peptide matches 610. Altogether, the data conditioning parameters are selected or chosen at 1150 and iteratively sequenced to the system resource manager 1140 for each of the scoring runs conducted by the scoring engine 1142 in a manner previously discussed.

The present invention has been particularly shown and described with respect to certain preferred embodiment(s). However, it will be readily apparent to those skilled in the art that a wide variety of alternate embodiments, adaptations or variations of the preferred embodiment(s), and/or equivalent embodiments may be made without departing from the intended scope of the present invention as set forth in the appended claims. Accordingly, the present invention is not limited except as by the appended claims.

1. A method for matching a sample analyzed by a mass spectrometer to a peptide in a database of peptides, comprising:
identifying a criterion for a successful peptide match; and
in a computing system environment, determining whether said criterion is met.

2. The method of claim 1, wherein said determining further includes assessing whether an algorithm score meets a threshold score.

3. The method of claim 1, wherein said determining further includes assessing whether a peptide coverage meets a threshold percent.

4. The method of claim 1, wherein said determining further includes assessing whether a spectrum coverage meets a threshold amount.

5. The method of claim 1, wherein said determining further includes a de novo sequencing.

6. The method of claim 1, further including applying a spectrum data conditioning parameter to an output of said mass spectrometer.

7. The method of claim 6, wherein said applying said spectrum data conditioning parameter further includes removing one of a low intensity peak, a low mass peak and noise from said output.

8. The method of claim 1, further including applying a peptide data conditioning parameter to said database of peptides or individual peptides thereof.

9. The method of claim 8, wherein said applying said peptide data conditioning parameter further includes selecting one of a taxonomy, a mass modification and an alternate digestion.

10. The method of claim 1, wherein said identifying said criterion further includes indicating said criterion at a time before said mass spectrometer analyzes said sample.

11. A computer readable media having computer executable instructions for performing the steps of claim 1.

12. A method for identifying when a mass spectrum output has achieved a successful correlation to a peptide in a database of peptides, comprising:
identifying a criterion for said successful correlation;
conducting a plurality of scoring algorithm analyses; and
in a computing system environment, determining whether said criterion is met after each of said plurality of scoring algorithm analyses.

13. The method of claim 12, further including modifying an initial parameter used in said conducting said scoring algorithm analyses.

14. The method of claim 12, further including stopping said conducting said plurality of scoring algorithm analyses upon said criterion being met.

15. The method of claim 12, wherein said identifying further includes receiving an indication of one of a threshold algorithm score, a threshold peptide coverage percentage, a threshold spectrum coverage amount, and a de novo sequencing.

16. The method of claim 12, wherein said conducting further includes changing a first scoring algorithm to a second scoring algorithm.

17. A computer readable media having computer executable instructions for performing the steps of claim 12.

18. A method for identifying when a scoring algorithm that correlates a mass spectrum output to a plurality of peptides in a database of peptides has made a successful correlation, comprising:
identifying a criterion for said successful correlation;
thereafter, conducting a plurality of scoring algorithm analyses, a first of said scoring algorithm analyses being conducted with a plurality of initial parameters;
thereafter, modifying one of said initial parameters for a second of said scoring algorithm analyses; and
in a computing system environment, determining whether said criterion is met after each of said plurality of scoring algorithm analyses.

19. The method of claim 18, further including stopping said conducting said plurality of scoring algorithm analyses upon said criterion being met.

20. The method of claim 18, further including receiving an indication of a plurality of spectrum data conditioning parameters to be applied to said mass spectrum output, said initial parameters including said spectrum data conditioning parameters.

21. The method of claim 18, further including receiving an indication of a peptide data conditioning parameter to be applied to said peptides or said database of peptides, said initial parameters including said peptide data conditioning parameters.

22. The method of claim 18, wherein said conducting further includes changing a first scoring algorithm to a second scoring algorithm.

23. A computer readable media having computer executable instructions for performing the steps of claim 18.

24. An in silico method for identifying when a scoring algorithm that correlates a mass spectrum output to a plurality of peptides in a database of peptides has made a successful correlation, said mass spectrum output corresponding to a sample analyzed by a mass spectrometer, comprising:
receiving an indication of a plurality of spectrum data conditioning parameters to be applied to said output;
receiving an indication of a plurality of peptide data conditioning parameters to be applied to said peptides or said database of peptides;
receiving an indication of criteria for said successful correlation;
conducting a scoring algorithm analysis according to a plurality of initial parameters of said peptide and spectrum data conditioning parameters;
determining whether a criterion of said criteria is met;
modifying one of said initial parameters; and
conducting another scoring algorithm analysis according to said modified said one of said initial parameters.

25. The method of claim 24, wherein said receiving an indication of criteria further includes receiving an indication of one of a threshold algorithm score, a threshold peptide coverage percentage, a threshold spectrum coverage amount, and a de novo sequencing.

26. The method of claim 24, wherein said receiving an indication of said spectrum data conditioning parameters further includes receiving an indication on removing one of a low intensity peak, a low mass peak and noise from said output.

27. The method of claim 24, wherein said receiving an indication of said peptide data conditioning parameter further includes receiving an indication of one of a taxonomy, a mass modification and an alternate digestion.

28. The method of claim 24, further including meeting said criterion of said criteria.

29. The method of claim 24, wherein said receiving said indication of said criteria further includes receiving said criteria at a time before said mass spectrometer analyzes said sample.

30. A computer readable media having computer executable instructions for performing the steps of claim 24.

31. In a computing system environment having a graphical user interface including a display and a user interface selection device, a method comprising:
displaying criteria indicative of a successful correlation between a mass spectrometer output and a plurality of peptides in a database of peptides; and
receiving an indication of a threshold score of a scoring algorithm that performs said correlation, a threshold peptide coverage percentage, a threshold spectrum coverage amount, or a de novo sequencing.

32. The method of claim 31, further including displaying and receiving an indication of a spectrum data conditioning parameter to be applied to said mass spectrometer output.

33. The method of claim 31, further including displaying and receiving an indication of a peptide data conditioning parameter to be applied to said peptides or said database of peptides.

34. A computing system environment, comprising an architecture having local or remote access to (i) a plurality of computer executable instructions for selecting a plurality of initial parameters of a scoring algorithm that correlates a mass spectrometer output with a plurality of peptides in a database of peptides; (ii) a plurality of computer executable instructions for modifying said initial parameters; (iii) a plurality of computer executable instructions for conducting a plurality of scoring algorithm analyses; and (iv) a plurality of computer executable instructions for indicating a successful correlation between said mass spectrometer output and said peptides.

35. The computing system environment of claim 34, wherein said architecture further includes a system resource manager having a local or remote access to a scoring engine that conducts said scoring algorithm analyses and criteria for said indicating said successful correlation.

36. The computing system environment of claim 34, wherein each of said plurality of computer executable instructions are obtained from a computer readable media.

37. A method for identifying a successful correlation of a mass spectrometer output with an amino acid sequence or a peptide in a database, comprising:
identifying a criterion for said successful correlation;
conducting a plurality of scoring algorithm analyses; and
in silico, determining whether said criterion is met.

38. An in silico method for iterating correlations of a mass spectrometer output with amino acid sequences or peptides in a database of same, comprising:
conducting a first scoring algorithm analysis in accordance with a first scoring algorithm and a plurality of initial parameters; and
changing said initial parameters into modified parameters or said first scoring into a second scoring algorithm.

39. The method of claim 38, further including conducting a second scoring algorithm analysis after said changing.

40. The method of claim 39, further including identifying a criterion for a successful correlation between said output and said amino acid sequences or peptides.

41. The method of claim 40, further including determining whether said criterion is met after each said first and second scoring algorithm analysis.

42. A computer readable media having computer executable instructions for performing the steps of claim 41.

43. An in silico method for iteratively correlating a mass spectrometer output with amino acid sequences or peptides in a database of same, comprising:
identifying a criterion for a successful correlation between said output and said amino acid sequences or peptides;
conducting a first scoring algorithm analysis in accordance with a first scoring algorithm and a plurality of initial parameters;
changing said initial parameters into modified parameters or said first scoring into a second scoring algorithm;
conducting a second scoring algorithm analysis after said changing; and
indicating said successful correlation upon said criterion being met.
